(19) United States
(12) Patent Application Publication
(10) Pub. No.: US 2026/0191473 A1
(43) Pub. Date: Jul. 9, 2026

(54) METHOD FOR PRE-DIAGNOSIS IN EMERGENCY SITUATIONS USING ARTIFICIAL INTELLIGENCE

(71) Applicants: Adrien RICCI, Allauch (FR); Christophe HAUS, Nice (FR); Maxime MONFORT, Paris (FR); Timothée CARGILL, Paris (FR)

(72) Inventors: Adrien RICCI, Allauch (FR); Christophe HAUS, Nice (FR); Maxime MONFORT, Paris (FR); Timothée CARGILL, Paris (FR)

(21) Appl. No.: 19/134,232

(22) PCT Filed: Jun. 8, 2023
(86) PCT No.: PCT/FR2023/000115

§ 371 Date: May 30, 2025

(51) Int. Cl. A61B 5/00; A61B 5/16; G06V 10/70; G06V 20/50; G06V 40/16; G16H 50/20

(52) U.S. Cl. CPC A61B 5/7267; A61B 5/0077; A61B 5/165; A61B 5/747; G06V 10/70; G06V 20/50; G06V 40/174; G16H 50/20; A61B 2505/01; A61B 2576/00; G06V 2201/03

(57) ABSTRACT

The present invention relates to a pre-diagnosis method in emergency situations based on Computer Vision artificial intelligence. By analyzing images transmitted by callers' smartphones, a pre-diagnosis is provided to emergency call center operators, following three major axes: traumatology, nature of the emergency situation, and patients' emotions. The method allows evaluating the nature and criticality of the patient's injuries, determining the most appropriate emergency service according to the emergency context, estimating the level of fear and stress of patients. The innovative method automatically qualifies, analyzes, sorts, and pre-diagnoses emergency calls based on images transmitted during emergency situations. Beyond use to improve everyday emergency diagnosis, the solution becomes a vital tool in the face of large-scale crises generating a number of emergency calls impossible to handle with the human resources of call centers.

The present invention relates to a pre-diagnosis method in emergency situations based on “Computer Vision” artificial intelligence. By analyzing images transmitted by callers' smartphones (using an application or cloud service), a pre-diagnosis is provided to emergency call center operators (EMS/Fire Department/Police), following three major axes: traumatology, nature of the emergency situation, and patients' emotions. (“HighWind AI”)

In France, approximately 69 million emergency calls per year are made to EMS, Fire Departments, and Police. They are based on GSM technology (voice telephone calls) that has not undergone major evolution since the introduction of 112 in 1997 with the first mobile phones. The few initiatives using smartphone capabilities operate only at the local level, mainly for including GPS position or video conference links transmitted by SMS. This lack of technology in emergency calls results in numerous errors and underestimations of the situation's severity, leading to many deaths each year. Indeed, the emergency operator must assess the criticality of a situation based on the caller's oral description, lasting between 10 to 15 minutes out of the 60 minutes that make up the “Golden Hour” for saving a life during a serious emergency. Moreover, in the face of large-scale crisis situations (natural or industrial disasters, terrorist attacks, fires, etc.), emergency call services are overwhelmed by the exponential number of calls (>100 calls per minute compared to about 2 per minute in normal times) and can no longer distinguish the criticality and nature of the calls. These crisis calls are generated not only by patients closest to the epicenter of the crisis but also by surrounding people who are not facing imminent danger, with the current method of emergency call sorting not allowing them to be distinguished without spending significant time exchanging with each caller individually.

The method according to the present invention remedies this disadvantage. Indeed, HighWind, a French startup, has developed an emergency call solution allowing the target population to transmit photos, videos, GPS position, pre-filled information, and VolP of an emergency situation via their smartphones, in addition to the traditional call, to emergency call centers equipped with a HighWind interface.

The solution operates at several levels:
Public: within the caller's mobile phone, through a smartphone emergency call application or a cloud interface opened by SMS link.
HighWind AI: analysis of data to provide a pre-diagnosis of the emergency call through the HighWind method using “Computer Vision” artificial intelligence.
Emergency call centers: via a dedicated interface (software or web) allowing emergency call operators to visualize information sent by callers, enriched by HighWind's AI analysis. HighWind therefore provides an emergency call service (“HighWind Service”) through an emergency call solution (“HighWind Solution”) that integrates all elements of transmission, analysis, and visualization of emergency information, from the caller through their smartphone (“HighWind Application”) to the emergency call center that receives it (“HighWind HQ Interface”) pre-analyzed by artificial intelligence thanks to the innovative technological method developed (“HighWind A!”).

Whether for a public or private population (e.g., in a company), the emergency call application or HighWind's cloud service is always distributed free of charge. The HighWind Application also allows, thanks to its internal artificial intelligence, to call emergency services worldwide, whether or not the emergency call center is equipped with the HighWind HQ Interface.

The HighWind Solution exploits the tunnel effect experienced by callers during an emergency situation, the restriction of their visual field pushing them to create a Situation-Phone-Eye alignment that allows taking pictures (main and rear “selfie”) with a single click on a single button of the HighWind emergency call Application.

The objective of the HighWind Solution is to increase patients' chances of survival, in public life or in business, by significantly improving the precision, accuracy, and speed of emergency diagnoses. Facing emergency calls lasting on average 10 to 15 minutes within the 60 minutes of the “Golden Hour” of serious emergencies (where the risk of death increases exponentially with passing time), the HighWind Solution is capable, by reducing the duration of an emergency call, of improving patients' chances of survival. The present invention relates to a pre-diagnosis method in emergency situations, based on the rapid retrieval and processing by “Computer Vision” artificial intelligence of emergency situation images provided by callers' smartphones or other capture means. Said method delivers an analysis following three major axes:
a. The nature and criticality of the traumatology;
b. The nature of the emergency situation associated with determining the most appropriate emergency service;
c. The estimation of emotions felt by the patient.

The pre-diagnosis method allows providing pre-diagnosed emergency situation images to an emergency reception center (public: emergency call centers such as EMS/Police/Fire Departments, but also private: in companies or as a remote solution during major events), and sending analyzed elements enabling determination, sorting, redirection, and decision support for the emergency operator to improve patient care and chances of survival.

As a result, the pre-diagnosis method significantly reduces the time needed to understand an emergency situation, in an automated manner or for the benefit of emergency operators.

The annexed drawings illustrate the present invention:

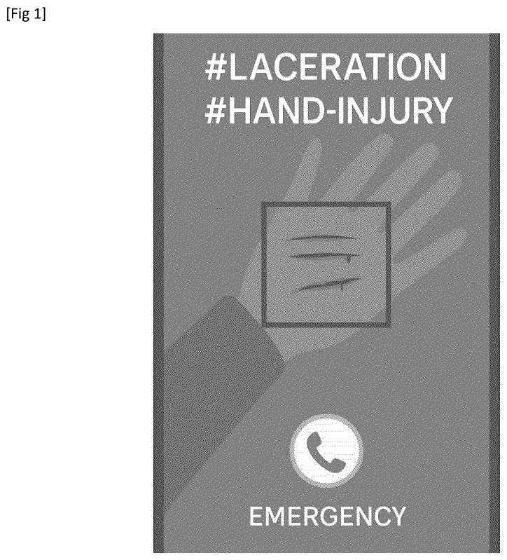

FIG. 1 is a visual of the HighWind Application concept distributed to the population.

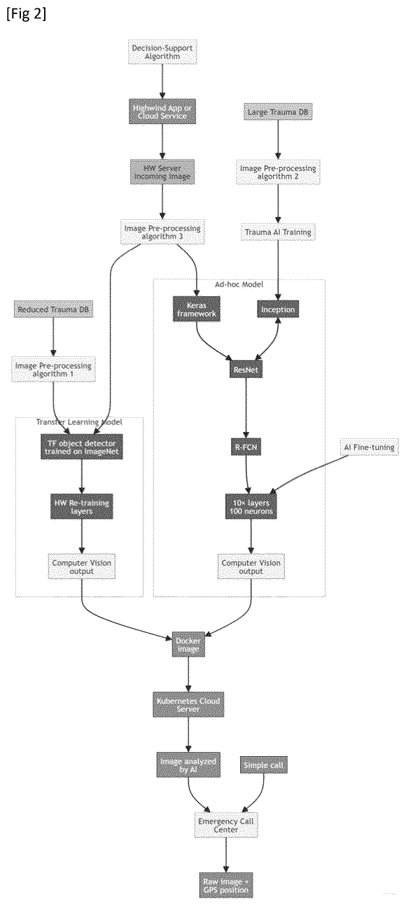

FIG. 2 is a visual of the type of architecture deployed at the heart of the method. Said architecture allowing the delivery of the HighWind Service is multiple and evolving, but follows the guiding thread of analysis by emergency pre-diagnoses using “Computer Vision” artificial intelligence. As an illustration, the model currently uses a dual architecture featuring different means based on “Computer Vision” and put at the service of the HighWind Solution.

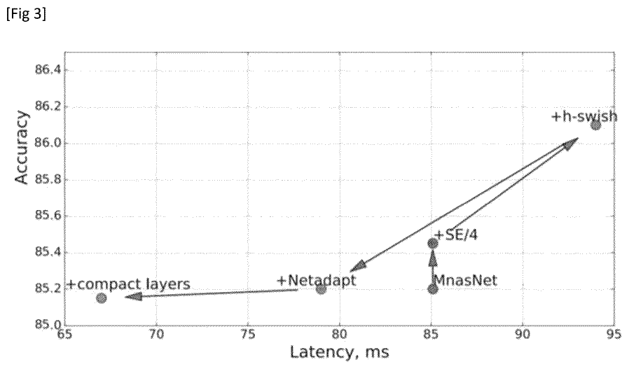

FIG. 3 is a graph illustrating the performance of the method, and more precisely the fact that the present invention secures a pre-diagnosis on the nature and criticality of a traumatological injury at 85% in less than 90 ms for open wounds. By comparison, it takes: 50 ms for the eye to linger on an image for it to be transmitted to the human brain; 300 ms for brain activity to increase considerably to analyze an image; several seconds or even minutes to determine the nature of a medical image; 10 to 15 minutes currently to provide an emergency call diagnosis with EMS telephone means.

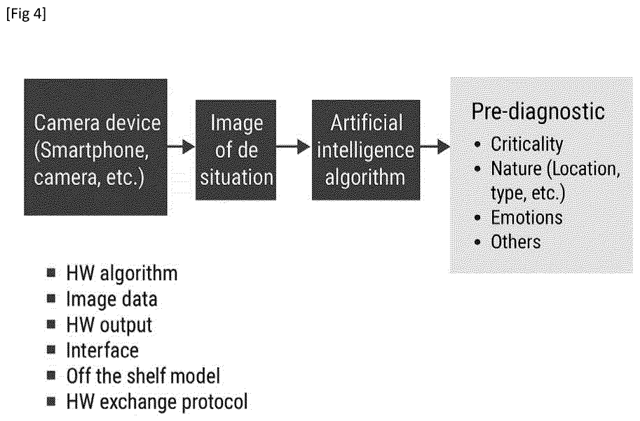

FIG. 4 illustrates the pre-diagnosis chain enabled by the present invention (first step).

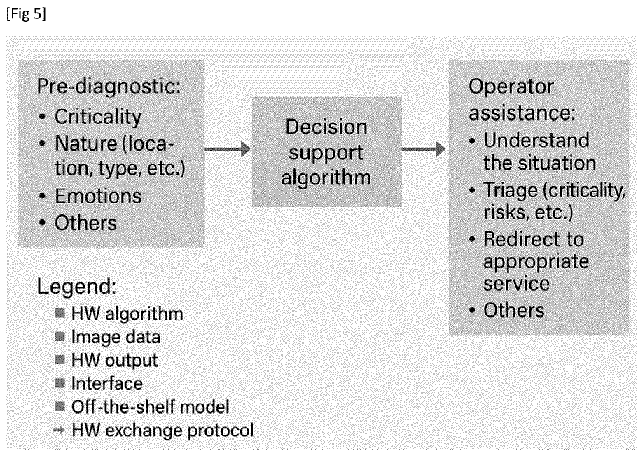

FIG. 5 illustrates the decision chain enabled by the present invention (second step).

In its current form, the step-by-step operation of the HighWind Application is as follows. It has been established through feedback from emergency personnel, patients, and survivors of major crises (FIGS. 4 and 5):
1. [Human] Open the application
Hidden [App] Confirms the region/country
Hidden [Brain reflex] eye-phone-situation alignment
2. [Human] Click on the call button
Hidden [App] Evaluates network throughput (2G to 5G)
Hidden [App] Checks whether the EMS center is equipped with HighWind or not
Hidden [App] Sends GPS position and pre-filled information
Hidden [App] Takes a main photo
Hidden [App] Takes a front photo (selfie)
Hidden [App] Sends photos to HighWind and EMS
Hidden [App] Calls 911/112 or equivalent depending on the country
Hidden [App] EMS receives pre-diagnosed images
3. [Human] Talk to EMS on the phone

Caller behavior has been tested and with feedback from emergency responders, due to the tunnel vision effect and the need for vigilance on the situation, the brain mechanism always produces an alignment: Eye—Phone—Situation when dialing or using the HighWind Application. (FIG. 1)

The unique and innovative character of the HighWind Service lies in its emergency pre-diagnosis system.

HighWind AI defines the process of retrieving emergency situation images transmitted by the caller's smartphone, analyzing them using “Computer Vision” artificial intelligence, and transmitting the analysis results and pre-diagnoses to emergency call centers (public, private, or any means of retrieving emergency calls). (FIGS. 4 and 5) The method allows analysis according to 3 defined pillars:
Traumatology: it identifies the type of injury and its level of criticality, particularly recognizing hemorrhage, lacerations, bullet impacts, burns (fire, acid, electricity), open fractures, dislocations, ulcers, hypothermia, etc.
Emergency situations: it recognizes the nature of the emergency situation and determines the most appropriate emergency service, distinguishing for example: road accidents, physical injuries, fire, landslide, earthquake, flood, etc.
Context & emotions: it determines patients' feelings, particularly in terms of pain, fear, stress, muscle tension, lying body, etc.

The results of the data analyzed according to the three major axes thus allow, through artificial intelligence, according to the interests of emergency services, to provide different types of recommendations, analyses, and emergency decision support, which may include, for example, but are not limited to: the criticality of the patient's condition, the most appropriate emergency service, risks to patients and rescuers (ballistic, blunt, natural, fire, slides, etc.), the nature of the present danger, the priority among different situations reported by callers, recommendations regarding the most appropriate equipment for intervention.

The three pillars pave the way for a fourth pillar allowing the automation of emergency call transcription and their future automated translation. Visually understanding the situation makes it easier to determine the lexicon that will be used in a given language and significantly increase the efficiency of the technology.

Within the framework of the problem of grouping emergency call numbers around a single number, brought to the National Assembly as part of a bill, the 2nd pillar of HighWind AI technology, determining the nature of the emergency situation, allows by extension to determine which emergency service is most suitable for a situation (EMS, fire department, police . . . ).

The artificial intelligence at the center of the present invention is of the “Computer Vision” type, exploiting “Deep-Learning” training on medical databases, emergency situation images, and the use of the HighWind Solution taking into account GDPR considerations.

The founding team of HighWind AI has been working since mid-2019, following exchanges with SAMU 75, SAMU 06, and SDIS 13 Firefighters, on the development of a smartphone emergency call solution allowing communication of: photos, GPS position, video, and VolP to EMS centers equipped with the HighWind Solution. The present invention is therefore intended to serve emergency service operators (EMS, fire departments, approved civil security associations, or companies specialized in telemedicine and emergency assistance for businesses), via the HighWind Solution.

1. Method of assistance in triage and pre-diagnosis during emergency situations for emergency call reception and regulation centers such as EMS, firefighters, and police, based on the retrieval and processing by “Computer Vision” artificial intelligence of emergency situation images provided by callers' smartphones or other capture means, followed by the analysis of data collected by artificial intelligence algorithm, to determine the nature and criticality of the emergency situation. Said method is characterized by the following steps:
Image capture by web application or downloaded application;
Rapid retrieval and processing of emergency situation images by “Computer Vision” artificial intelligence, exploiting “Deep-Learning” training on medical databases, emergency situation images, and seeking to recognize elements according to 3 key axes:
TRAUMATOLOGY: it identifies the type of injury and its level of criticality, particularly recognizing hemorrhage, lacerations, bullet impacts, burns from fire, acid, or electricity, open fractures, dislocations, ulcers, hypothermia, etc.
SITUATION AND/OR CONTEXT: it recognizes the nature of the emergency situation, for example: road accident, physical injuries, fire, landslide, earthquake, flood, etc.
EMOTIONS: it quantifies the level of pain expressed by the patient via “Deep-Learning” training on “selfie” faces from medical databases, emergency situation images.
Analysis of data by artificial intelligence, of data from “Computer Vision”, allowing to provide a pre-diagnosis of the nature and criticality of the emergency call.
Visualization of information sent by callers, enriched with analysis by “Computer Vision” artificial intelligence, within the emergency call center allowing triage, redirection, decision support for the emergency operator, display of risks to patients and rescuers, and priority among different situations reported by callers, to improve patient care and chances of survival.

2. Method of assistance in triage and pre-diagnosis during emergency situations for emergency call reception and regulation centers such as EMS, firefighters, and police, based on the retrieval and processing by “Computer Vision” artificial intelligence of emergency situation images provided by callers' smartphones or other capture means, followed by the analysis of data collected by artificial intelligence algorithm, to determine the nature and criticality of the emergency situation, according to claim 1, characterized by:
Determining the most appropriate emergency service according to their activity, EMS, firefighters, police, by retrieving and processing by “Computer Vision” artificial intelligence of emergency situation images provided by callers' smartphones or other capture means, via the 2nd detection axis of claim 1 “situation and/or context”, which allows recognizing through “Deep-Learning” training the nature of the emergency situation such as a road accident, physical injuries, a fire, a landslide, an earthquake, a flood.
